(19) United States
(12) Patent Application Publication
(10) Pub. No.: US 2004/0054403 A1
(43) Pub. Date: Mar. 18, 2004

(54) Angular orientation of a stent

(76) Inventor: Henry M. Israel, Bnei Brak (IL)

Correspondence Address: Dekel Patent Ltd., Beit HaRofim, 18 Menuha VeNahala Street, Room 27, Rehovot, IL

(21) Appl. No.: 10/245,577

(22) Filed: Sep. 18, 2002

(51) Int. Cl. A61F 2/06

(52) U.S. Cl. 623/1.35

(57) ABSTRACT

A stent assembly comprising at least two markers located at different angular orientations on the stent assembly, the markers being discernible by an imaging device, wherein a projection of the at least two markers on an imaging plane uniquely defines an angle of rotation of the stent assembly about an axis thereof with respect to a reference.

### FIELD OF THE INVENTION

The present invention relates generally to stents, and particularly to determining the angular orientation of a stent, e.g., a bifurcated stent, in a body lumen.

### BACKGROUND OF THE INVENTION

A stent is a well known device used to support an intraluminal wall, used in procedures, such as but not limited to, percutaneous transluminal coronary angioplasty (PTCA). Various types of stent architectures are known in the art, including braided stents (filaments or wires, wound or braided into a particular configuration), or mesh stents (metal mesh bent or formed into a particular shape), among others.

Typically, a stent may be restrained in a radially compressed configuration by a sheath or catheter, and delivered by a deployment system or “introducer” to the site where it is required. The introducer may enter the body through the patient's skin, or through a blood vessel exposed by minor surgical means. When the introducer has been threaded into the body lumen to the stent deployment location, the introducer is manipulated to cause the stent to be released. The stent expands to a predetermined diameter at the deployment location, and the introducer is withdrawn. Stent expansion may be effected by spring elasticity, balloon expansion, or by the self-expansion of a thermally or stress-induced return of a shape memory alloy (such as a nickel-titanium alloy, e.g., NITINOL) to a pre-conditioned expanded configuration.

The travel of the stent through the body vessels to the stent deployment location may be imaged by various techniques, such as but not limited to, fluoroscopy. However, imaging equipment can generally map the travel of the stent in only one plane. This provides an inadequate amount of information regarding the spatial location and angular orientation of the stent. For deployment in bifurcated lumen, such as but not limited to, the carotid artery, it is critical to orient the stent properly to match the spatial orientation of the bifurcation. The lack of three-dimensional information makes this orientation difficult and cumbersome guesswork.

### SUMMARY OF THE INVENTION

The present invention seeks to solve the abovementioned problem of the prior art, and to provide a stent whose angular orientation in three-dimensional space may be easily determined. In an embodiment of the present invention, the stent may be provided with markers discernible by an imaging device. The projections of the markers on an imaging plane uniquely define the angular orientation of the stent with respect to a defined reference. The three-dimensional position and orientation of the stent may thus be determined by the two-dimensional projection of the markers on the imaging plane.

The present invention also provides a novel bifurcated stent construction with a tapered nozzle and branch, which may or may not be off-center with the main body portion of the stent. The bifurcated stent may be constructed from a mesh pattern.

There is thus provided in accordance with an embodiment of the present invention a stent assembly comprising at least two markers located at different angular orientations on the stent assembly, the markers being discernible by an imaging device, wherein a projection of the at least two markers on an imaging plane uniquely defines an angle of rotation of the stent assembly about an axis thereof with respect to a reference. The projection of the at least two markers on an imaging plane may uniquely define an angle of rotation of the stent assembly about a longitudinal axis thereof with respect to the reference.

In accordance with an embodiment of the present invention the at least two markers are orthogonally spaced from one another on the stent assembly.

Further in accordance with an embodiment of the present invention the projection of the at least two markers on an imaging plane uniquely defines at least four mutually orthogonal angles of rotation of the stent assembly about an axis thereof with respect to the reference.

In accordance with an embodiment of the present invention the stent assembly comprises a body portion and a bifurcation extending from the body portion.

Further in accordance with an embodiment of the present invention the stent assembly comprises at least one of a stent, a sheath, a catheter and an introducer, and the markers are formed on a portion of at least one of the stent, the sheath, the catheter, the introducer, the body portion and the bifurcation.

Still further in accordance with an embodiment of the present invention the bifurcation comprises a tapered extension of the body portion and a branch extended at an angle to the tapered extension.

In accordance with an embodiment of the present invention the branch is placed off-center with respect to the body portion. Alternatively, the branch may extend from a centerline of the body portion.

Further in accordance with an embodiment of the present invention the body portion and the tapered extension are formed from a mesh pattern. The body portion and the tapered extension may be formed with an aperture, and the branch may be secured to the body portion at the aperture.

In accordance with an embodiment of the present invention the mesh pattern comprises a plurality of sub-sections of generally equal length.

Further in accordance with an embodiment of the present invention the mesh pattern comprises a first plurality of sub-sections and a second plurality of sub-sections, wherein the first sub-sections are shorter than the second sub-sections.

Still further in accordance with an embodiment of the present invention the second sub-sections may be arranged in a straight pattern or in a curved pattern.

Additionally in accordance with an embodiment of the present invention the mesh pattern further comprises an additional pattern with an aperture formed therein.

In accordance with an embodiment of the present invention at least one additional set of markers may be provided on the stent assembly angularly spaced from one another. The at least one additional set of markers may be angularly spaced from the at least two markers and/or may be placed on a different portion of the stent assembly than the previously mentioned markers, which may provide better accuracy in determining the angular orientation of the stent assembly or portions thereof or the angular orientation of different portions of the stent assembly with respect to each other.

There is also provided in accordance with an embodiment of the present invention a method for determining an angular orientation of a stent in a body lumen, the method comprising inserting a stent assembly into a body lumen, the stent assembly comprising at least two markers located at different angular orientations on the stent assembly, the markers being discernible by an imaging device, and sensing and processing a projection of the at least two markers on an imaging plane, wherein the projection uniquely defines an angle of rotation of the stent assembly about an axis thereof with respect to a reference. A contrast agent may be passed through the stent assembly.

### BRIEF DESCRIPTION OF DRAWINGS

The present invention will be further understood and appreciated from the following detailed description taken in conjunction with the drawing in which:

FIGS. 1A and 1B are simplified side-view and end-view illustrations, respectively, of a stent, constructed and operative in accordance with an embodiment of the invention;

FIG. 2 is a simplified illustration of the stent of FIGS. 1A and 1B introduced into a body lumen, and showing a projection of markers on the stent projected on an imaging plane, in accordance with an embodiment of the invention;

FIGS. 3A-3C are simplified pictorial illustrations of one construction of the stent of FIGS. 1A and 1B, in accordance with an embodiment of the invention, comprising a bifurcation with a tapered extension and a branch extending off-center with respect to a body portion of the stent;

FIG. 3D is a simplified pictorial illustration of another construction of the stent of FIGS. 1A and 1B, in accordance with another embodiment of the invention, wherein the branch may extend from a centerline of the body portion; and

FIGS. 4A-4D are simplified illustrations of mesh patterns for forming a stent, wherein FIG. 4A illustrates a pattern which may be used to form a straight stent, FIG. 4B illustrates a pattern which may be used to form a stent with a straight taper, FIG. 4C illustrates a pattern which may be used to form a stent with an offset taper, and FIG. 4D illustrates a pattern which may be used to form a stent with an aperture, in accordance with embodiments of the invention.

### DETAILED DESCRIPTION OF THE INVENTION

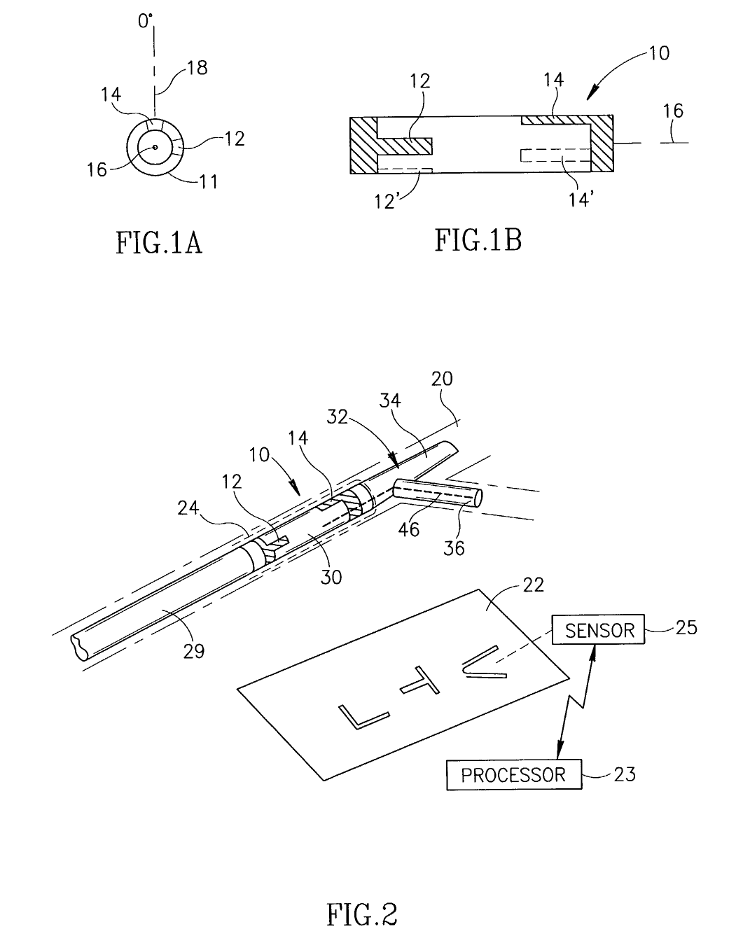

Reference is now made to FIGS. 1A and 1B, which illustrate a stent assembly 10, constructed and operative in accordance with an embodiment of the invention.

Stent assembly 10 comprises two or more markers 12 and 14 located at different angular orientations on stent assembly 10, such as on a stent 11. Markers 12 and 14 may be discernible by an imaging device, such as but not limited to, a fluoroscope (not shown). In FIGS. 1A and 1B, markers 12 and 14 are spaced axially from one another, although they do not have to be. In one embodiment of the invention, markers 12 and 14 are orthogonally spaced from one another on stent assembly 10, that is, angularly spaced 90° apart. In another embodiment of the invention, another set of markers 12′ and 14′ may be provided on stent assembly 10, as indicated in broken lines in FIG. 1A. The additional set or sets of markers may be orthogonally spaced from one another. Alternatively, markers 12 and 14 and/or the additional set(s) of markers may be angularly spaced from one another at angles other than 90°. Use of multiple sets of markers may increase the accuracy of determining the angular orientation of the stent assembly 10, and reduce the need for interpolation between markers.

Markers 12 and 14 uniquely define an angle of rotation of stent assembly 10 about an axis 16 thereof with respect to a reference 18. (Axis 16 may be the longitudinal axis of stent assembly 10.) Referring to FIG. 1B, it is seen that at reference 18 (angular orientation of 0°), marker 12 is pointing to the right and marker 14 is pointing upwards. At the angular orientation of 90°, corresponding to 90° counterclockwise rotation of stent assembly 10 about its longitudinal axis 16 in the sense of FIG. 1B, marker 12 is pointing upwards and marker 14 is pointing to the left. At the angular orientation of 180°, corresponding to 180° rotation of stent assembly 10 about its longitudinal axis 16 in the sense of FIG. 1B, marker 12 is pointing to the left and marker 14 is pointing downwards. At the angular orientation of 270°, corresponding to 270° counterclockwise rotation of stent assembly 10 about its longitudinal axis 16 in the sense of FIG. 1B, marker 12 is pointing downwards and marker 14 is pointing to the right.

Reference is now made to FIG. 2, which illustrates the stent 11 of stent assembly 10 introduced into a body lumen 20. A projection of the markers 12 and 14 are projected on an imaging plane 22. It may be appreciated that the projection of markers 12 and 14 in this example is the same for both the upward and downward positions. Nevertheless, the combined projection of markers 12 and 14 on imaging plane 22 uniquely defines the angle of rotation of stent assembly 10 about axis 16 with respect to reference 18.

The following table summarizes the orthogonal positions of the markers and their projection on imaging plane 22 (the up and down projections are identical and are designated as ‘1’).

| Degrees of Rotation | Marker 12 | Marker 14 |
|---|---|---|
| 0 | Right | ‘1’ (Up) |
| 90 | ‘1’ (Up) | Left |
| 180 | Left | ‘1’ (Down) |
| 270 | ‘1’ (Down) | Right |

Accordingly, the projections of markers 12 and 14 on imaging plane 22 uniquely define at least four mutually orthogonal angles of rotation of stent assembly 10 about axis 16 with respect to reference 18. A processor 23 may process the encoded information as sensed at the imaging plane 22 by appropriate sensors 25 of an imaging system (not shown), in order to determine the angular orientation of stent assembly 10. For example, the combination of R1 means that stent assembly 10 is at 0°. The combination of 1R means that stent assembly 10 is at 90°, and so forth. Other angular orientations may be determined by interpolation between the four orthogonal angles. The markers may act as encoders from which the angular orientation of stent assembly 10 may be determined. The three-dimensional position and orientation of stent assembly 10 may thus be determined by the two-dimensional projection of the markers 12 and 14 on imaging plane 22.

As seen in FIG. 2, the stent 11 of stent assembly 10 may be disposed in a sheath or catheter 24 for delivery to a deployment site by an introducer 29. A bifurcation 32 may extend from body portion 30. Markers 12 and 14 may be formed on any portion of stent assembly 10. Accordingly, markers 12 and 14 may be formed, without limitation, on any portion of the stent 11, sheath or catheter 24, introducer 29, body portion 30 or bifurcation 32. For example, one set of markers may be placed on body portion 30 and another set of markers may be placed on bifurcation 32. In this manner, the angular orientation of bifurcation 32 with respect to that of body portion 30 may be easily determined by the sets of markers on each corresponding part of the stent assembly 10.

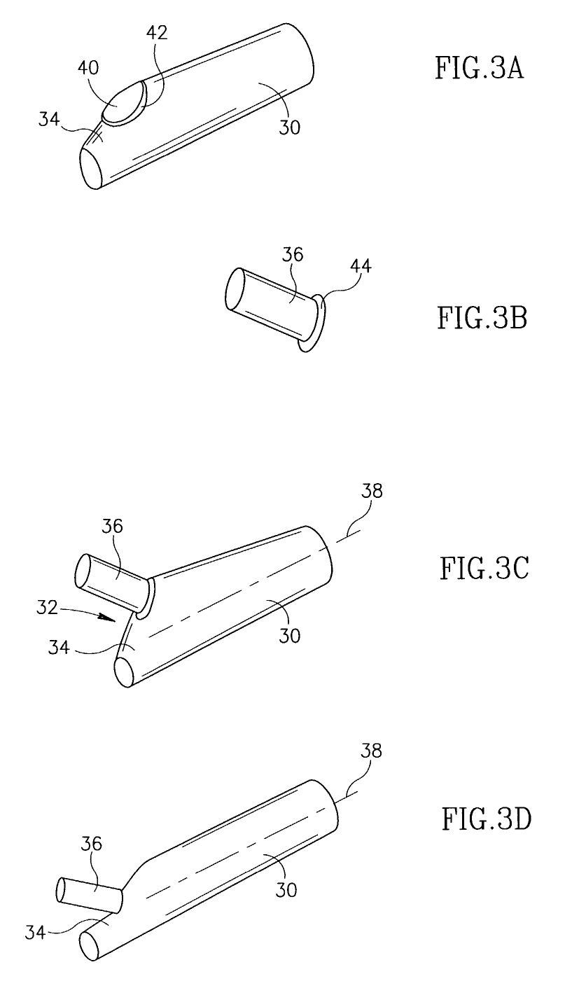

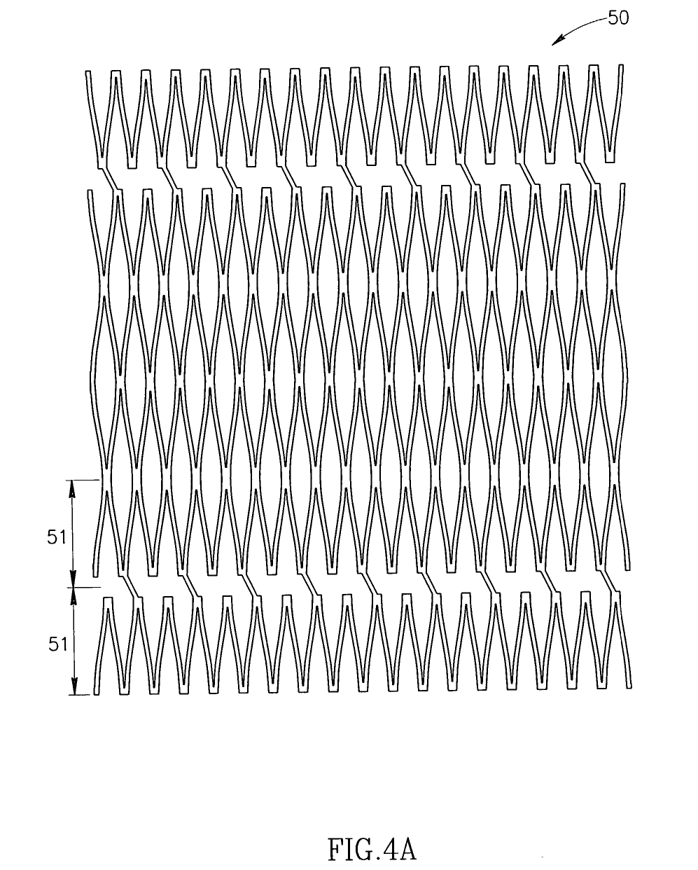

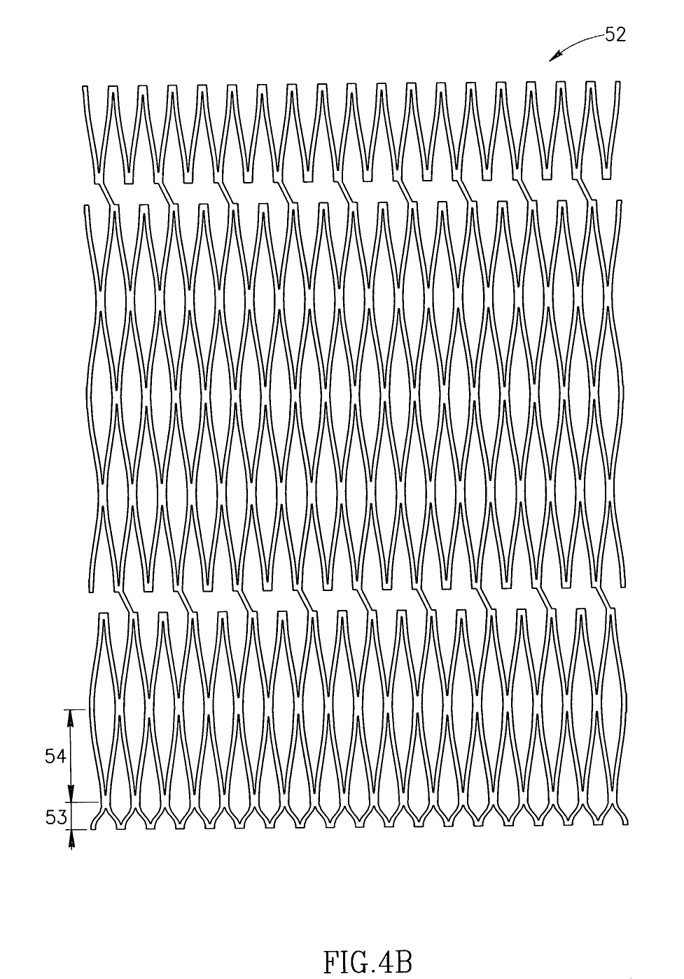

Reference is now additionally made to FIGS. 3A-3C, which illustrate one construction of the stent of stent assembly 10, in accordance with an embodiment of the invention. Bifurcation 32 may comprise a tapered extension 34 of body portion 30, and a branch 36 extended at an angle to tapered extension 34. Branch 36 may be placed off-center with respect to body portion 30. Alternatively, as shown in FIG. 3D, branch 36 may extend from a centerline 38 of body portion 30. As seen in FIG. 3A, body portion 30 and tapered extension 34 may be formed with an aperture 40. Aperture 40 may be formed with mounting structure 42, such as but not limited to, a rim. As seen in FIG. 3B, branch 36 may be formed with mounting structure 44, such as but not limited to, a flange. Branch 36 may be secured to body portion 30 at aperture 40, such as but not limited to, by mating (snapping, clinging, bonding, tight-fitting, or otherwise joining) the mounting structures of branch 36 and aperture 40 together.

For example, body portion 30 may be introduced to the stent deployment site without branch 36. Afterwards, branch 36 may be introduced and fed through body portion 30. Branch 36 may then be pushed through aperture 40 and snapped or otherwise fixed in place at aperture 40, thereby forming bifurcation 32 with tapered extension 34. During imaging, a contrast agent 46 may be disposed in (e.g., passed through) bifurcation 32 for imaging the projections of markers 12 and 14 on imaging plane 22, as described hereinabove.

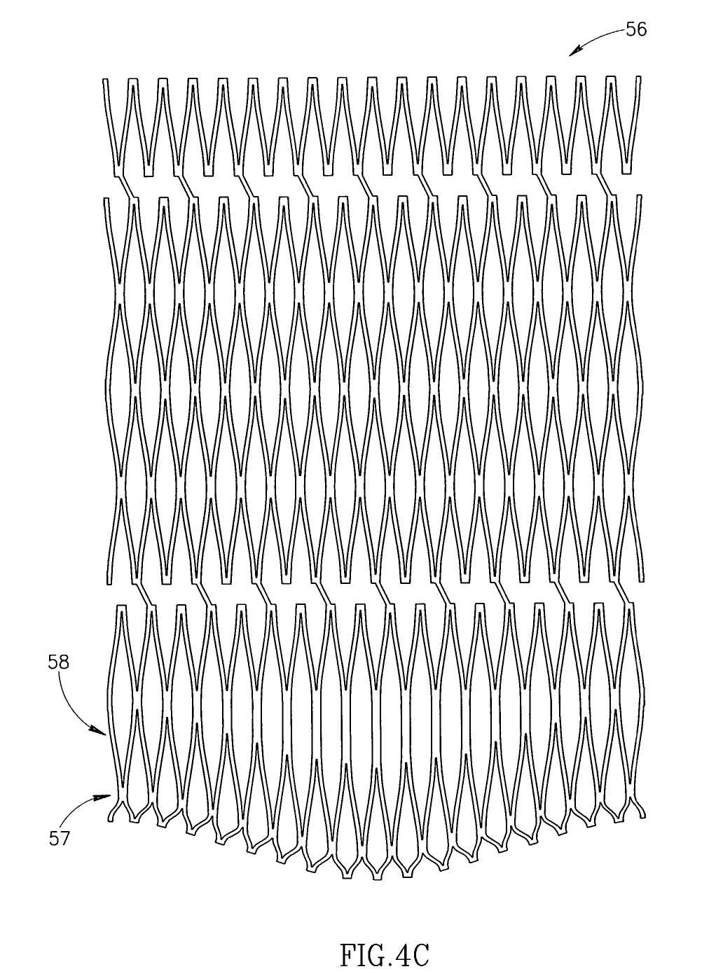

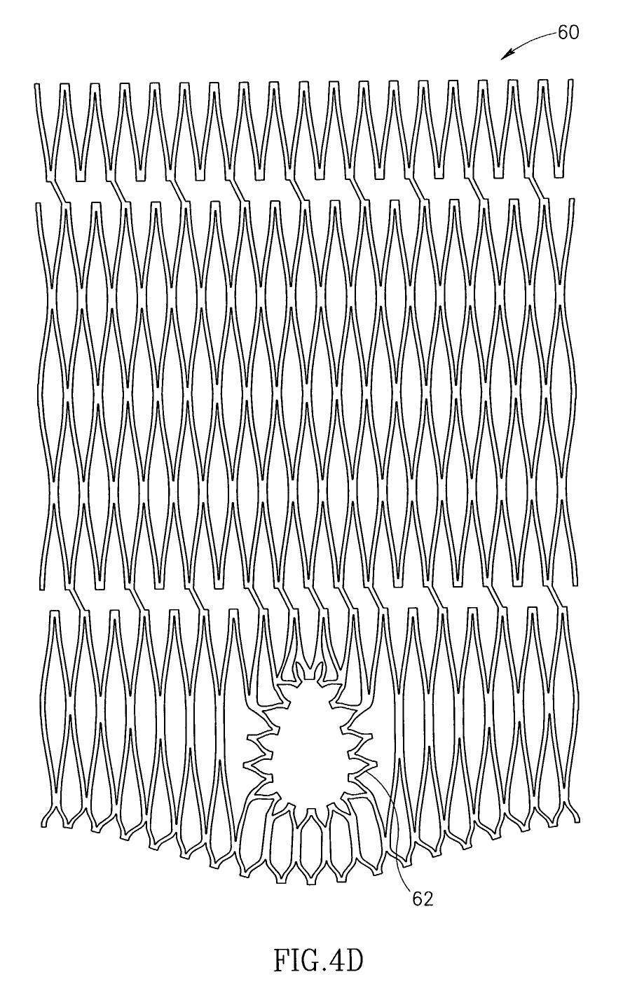

Reference is now made to FIGS. 4A-4D, which illustrate mesh patterns for forming a stent with a branch. Branch 36 may be made with a pattern 50 shown in FIG. 4A, which may be used to form a straight stent. Pattern 50 may be used as a cylindrical pattern with cylindrical coordinates ρθ to form the stent by any suitable method, such as but not limited to, laser etching or cutting. Pattern 50 may also be used as a flat pattern to form the stent by any suitable method, such as but not limited to, laser etching or cutting, followed by bending around a mandrel and welding a seam. Pattern 50 may comprise subsections 51 of generally equal length. A stent formed from pattern 50 may be straight due to the equal lengths of sub-sections 51.

FIG. 4B illustrates a mesh pattern 52, which may be used to form a stent with a straight taper. Pattern 52 may comprise a first plurality of sub-sections 53 and a second plurality of sub-sections 54, wherein first sub-sections 53 are shorter than second sub-sections 54. The first sub-sections 53 may form a generally straight cylindrical stent portion, whereas the second sub-sections 54, due to their shorter length, may form a straight taper extending from the straight cylindrical stent portion formed by first sub-sections 53. Such a stent construction may be used in the embodiment of FIG. 3D wherein branch 36 extends from centerline 38 of body portion 30.

FIG. 4C illustrates a mesh pattern 56, which may be used to form a stent with an offset taper. Pattern 56 may comprise a first plurality of sub-sections 57 and a second plurality of sub-sections 58, wherein first sub-sections 57 are shorter than second sub-sections 58. The second sub-sections 58 may be arranged in a curved pattern, unlike pattern 52 of FIG. 4B, wherein the second sub-sections 53 may be arranged in a straight pattern. The first sub-sections 57 may form a generally straight cylindrical stent portion, whereas the second sub-sections 58, due to their shorter length and curved pattern, may form a taper extending in an offset manner from the straight cylindrical stent portion formed by first sub-sections 57. Such a stent construction may be used in the embodiment of FIG. 3C wherein branch 36 is placed off-center with respect to body portion 30.

FIG. 4D illustrates a mesh pattern 60, which may be used to form a stent with an aperture, such as the embodiment of FIG. 3A, which includes body portion 30 and the tapered extension 34 formed with aperture 40. Pattern 60 may be identical to pattern 56, with an additional pattern 62 with an aperture formed therein. It is appreciated that the invention is not limited to the patterns shown in FIGS. 4A-4D.

It will be appreciated that the invention is not limited to what has been described hereinabove merely by way of example. Rather, the invention is limited solely by the claims that follow.

What is claimed is:

1. Apparatus comprising:
a stent assembly comprising at least two markers located at different angular orientations on said stent assembly, said markers being discernible by an imaging device, wherein a projection of said at least two markers on an imaging plane uniquely defines an angle of rotation of said stent assembly about an axis thereof with respect to a reference.

2. Apparatus according to claim 1, wherein the projection of said at least two markers on an imaging plane uniquely defines an angle of rotation of said stent assembly about a longitudinal axis thereof with respect to the reference.

3. Apparatus according to claim 1, wherein said at least two markers are orthogonally spaced from one another on said stent assembly.

4. Apparatus according to claim 1, wherein the projection of said at least two markers on an imaging plane uniquely defines at least four mutually orthogonal angles of rotation of said stent assembly about an axis thereof with respect to the reference.

5. Apparatus according to claim 1, wherein said stent assembly comprises a body portion and a bifurcation extending from said body portion.

6. Apparatus according to claim 5, wherein said stent assembly comprises at least one of a stent, a sheath, a catheter and an introducer, and said markers are formed on a portion of at least one of said stent, said sheath, said catheter, said introducer, said body portion and said bifurcation.

7. Apparatus according to claim 5, wherein said bifurcation comprises a tapered extension of said body portion and a branch extended at an angle to said tapered extension.

8. Apparatus according to claim 7, wherein said branch is placed off-center with respect to said body portion.

9. Apparatus according to claim 7, wherein said branch extends from a centerline of said body portion.

10. Apparatus according to claim 7, wherein said body portion and said tapered extension are formed from a mesh pattern.

11. Apparatus according to claim 7, wherein said body portion and said tapered extension are formed with an aperture, and said branch is secured to said body portion at said aperture.

12. Apparatus according to claim 10, wherein said mesh pattern comprises a plurality of sub-sections of generally equal length.

13. Apparatus according to claim 10, wherein said mesh pattern comprises a first plurality of sub-sections and a second plurality of sub-sections, wherein said first sub-sections are shorter than said second sub-sections.

14. Apparatus according to claim 13, wherein said second sub-sections are arranged in a straight pattern.

15. Apparatus according to claim 13, wherein said second sub-sections are arranged in a curved pattern.

16. Apparatus according to claim 10, wherein said mesh pattern further comprises an additional pattern with an aperture formed therein.

17. Apparatus according to claim 1, further comprising at least one additional set of markers angularly spaced from one another.

18. Apparatus according to claim 17, wherein said at least one additional set of markers is angularly spaced from said at least two markers.

19. Apparatus according to claim 17, wherein said at least one additional set of markers is placed on a different portion of said stent assembly than said at least two markers.

20. A method for determining an angular orientation of a stent in a body lumen, the method comprising:
inserting a stent assembly into a body lumen, said stent assembly comprising at least two markers located at different angular orientations on said stent assembly, said markers being discernible by an imaging device; and
sensing and processing a projection of said at least two markers on an imaging plane, wherein said projection uniquely defines an angle of rotation of said stent assembly about an axis thereof with respect to a reference.

21. The method according to claim 20, further comprising passing a contrast agent through said stent assembly.
